(19) United States
(12) Patent Application Publication
(10) Pub. No.: US 2004/0265277 A1
(43) Pub. Date: Dec. 30, 2004

(54) Agents for treating malignant diseases using the protein YB-1

(76) Inventor: Per Sonne Holm, Munchen (DE)

Correspondence Address: PENDORF & CUTLIFF, 5111 MEMORIAL HIGHWAY, TAMPA, FL, 33634-7356, US

(21) Appl. No.: 10/834,641

(22) Filed: Apr. 29, 2004

Related U.S. Application Data
(63) Continuation of application No. 10/029,574, filed on Dec. 20, 2001, now abandoned.

(30) Foreign Application Priority Data
Jun. 21, 1999 (DE) 199 29 569.7
Jun. 20, 2000 (WO) PCT/DE00/01978

(51) Int. Cl. A61K 48/00; C12N 7/00; C12N 15/861

(52) U.S. Cl. 424/93.2; 435/456; 435/235.1

(57) ABSTRACT

The invention concerns agents for the treatment of malignant diseases using the protein YB-1. It makes it possible to produce an E1A-independent replication of adenoviruses in tumour cells in order to destroy these tumour cells, and to destroy tumour cells which contain the protein YB-1 in the nucleus by using E1A-defective adenoviruses.

### BACKGROUND OF THE INVENTION

1. Field of the Invention

The present invention is related to methods for the replication of adenoviruses, the treatment of tumors and agents for such treatments.

2. Description of the Related Art

Today quite a number of different methods are used for the treatment of tumors. Apart from surgical methods chemotherapeutant and radiopharmaceuticals are used. However, all of theses techniques have more or less pronounced side effects.

A rather new technology uses replication selective viruses for the lysis of tumor cells. More precisely, the viral agent replicates only in tumor cells whereupon the infected cells undergo lysis and concomitantly release the virus which infects adjacent tumor cells. As the virus may replicate in tumor cells only non-tumor cells and non-tumor tissue remain unaffected. Examples for such replication selective viruses are gene attenuated adenoviruses and herpesviruses (Martuza, R. et al. Science 252, 854-858 (1991); Fueyo, J et al. Oncogene 19, 2-12 (2000)).

Adenovirus d1 1520 (also referred to as Onyx-015) is an example for such adenovirus which has been used in clinical trial phases I and II (Khuri, F. et al. Nature Medicine 6, 879-885 (2000). Onyx-015 is more particularly an adenovirus having the E1B 55 kDa gene deleted. The gene product of the E1B 55 kDa gene is involved in the inhibition of p53, the transport of viral mRNA and the inactivation of the host cell's protein synthesis.

P 53 which is encoded by TP53, triggers a complex regulatory mechanism (Zambetti, G. P. et al., FASEB J, 7, 855-865) which suppresses, among others, the efficient replication of viruses such as adenoviruses in a cell. However, TP 53 is either deleted or mutated in about 50% of all human tumors. Because of this there is no apoptosis upon chemotherapy or radiotherapy of this kind of tumors and thus the therapy unsuccessful.

### SUMMARY OF THE INVENTION

An objective of the present invention was therefore to provide means for a more efficient tumor therapy on the basis of virus induced tumor lysis.

In a first aspect the invention is related to an E1-deficient adenovirus, more particularly an E1A-deficient adenovirus, comprising a YB-1 encoding DNA sequence. In a second aspect the invention is related to a pharmaceutical composition comprising an E1-deficient adenovirus, more particularly an E1A-deficient adenovirus, comprising a YB-1 encoding DNA sequence, and at least a pharmaceutically acceptable carrier.

In a preferred embodiment the pharmaceutical composition is for the treatment of tumors.

In a third aspect the invention is related to the use of an E1-deficient adenovirus, more particularly an E1A-deficient adenovirus for the manufacture of a medicament for the treatment and/or prevention of tumors.

In a preferred embodiment the medicament comprises further compounds which damage tumor cells.

In a further embodiment the tumor cells exhibit YB-1 in the nucleus.

In a fourth aspect the present invention is related to the use of YB-1 and/or a nucleic acid coding for YB-1 for E1-independent replication, more particularly E1A-independent replication of adenovirus.

The present inventors have surprisingly found that E1-deficient and more precisely E1A-deficient adenoviruses are able to replicate in cells which contain the factor YB-1 in the nucleus. Because of this relationship it is possible to use E1-deficient, and more particularly E1A-deficient adenovirus to infect YB-1 expressing tumor cells and thus allowing for replication and thus lysis of the tumor. Since the virus only replicates in cells in which YB-1 is located in the nucleus, only these cells are destroyed by the virus.

The protein YB-1 is a member of the Y-box protein family which binds to the DNA sequence motif Y-box. The Y-box motif is a transcriptional regulatory element which is found in the promoter or enhancer regions of a number of different genes which play a role in the regulation of cell proliferation (Ladornery, M. et al., 1995, Bioassays 17:9-11; Didier, D. K. et al., 1988, PNAS, 85, 7322-7326).

The connection between YB-1 (factor YB-1) and MDR-1-gene expression (MDR multidrug resistance) and the location of YB-1 in the cell nucleus and P-glycoprotein (Pgp or P-170) have been examined by Bargou et al., (Bargou, R. C. et al., Nature Med. 3, 1997, 4:447-450). Analysis of mammary carcinoma tissue showed a significant overexpression of YB-1 compared to normal breast epithelium. In addition it was shown that after the factor YB-1 becomes localized in the nucleus, synthesis of the P-glycoprotein occurs and hence the formation of the multidrug resistance phenotype. Of all examined tumours 30% additionally had YB-1 in the nucleus. All of these (100%) expressed the P-glycoprotein.

YB-1 as used herein means any YB-1 molecule or a part thereof, such as, but not limited to an N- or C-terminal truncated version, which still allows the generation of the phenomenon described herein, namely to allow for a replication of an E1-deficient, more particularly E1A-deficient adenovirus in the presence of said YB-1 molecule in the nucleus of a cell.

As used herein, E1-deficient adenovirus means an adenovirus of which the proteins (E1A and/or E1B) of the E1gene are deficient. Such deficiency may be caused either by a defective translation or transcription or may be due to the absence of a part or the whole of the gene coding for E1 or being essential for its expression. In other words, E1-deficient may also be understood functionally such as no E1-protein(s) are produced by the respective E1-deficient adenovirus. More particularly, an E1-deficient adenovirus may also be an E1A-deficient adenovirus and the respective deficiency is a deficiency as described above in relation to the protein E1A, i.e. there is no functional E1A protein or no functional E1A protein level. The role of the protein E1A in adenovirus replication is described, among others, in Flint et al. (Flint, J. and Shenk, T. A., 1989, Rev. Genet., 23, 141-161) report on the role of the protein E1A in adenovirus replication.

Based on the surprising finding underlying the present invention, namely that E1A-deficient adenovirus may replicate in nucleus-exhibiting YB-1 tumor cells, it is possible to genetically engineer an E1-deficient adenovirus such as to incorporate a YB-1 encoding DNA sequence. The selection of a respective sequence is within the skills of the one of the art. YB-1 sequences may be taken from any respective data base. It is also within the skills of the one of the art to modify the adenovirus such as to express the YB-1 protein, more particularly to express YB-1 in the nucleus. Such adenovirus is thus a means or pharmaceutical for the therapy of tumors not exhibiting YB-1 in the nucleus per se.

Also based on the relationship between E1 deficiency and YB-1, the present inventors have perceived another strategy for the treatment of the tumors. Basically, this strategy is based on the observation that it is possible to transfer YB-1 from the cytoplasma into the nucleus. This may be done by radiation therapy, chemotherapy or hyperthermia or upon the influence of cytotoxic agents (Levenson, V. V., Davidovich, I. A., and Roninson, I. B. Pleiotropic resistance to DNA-interactive drugs is associated with increased expression of genes involved in DNA replication, repair, and stress response. Cancer Res., 60: 5027-5030, 2000). Under such conditions YB-1 is transferred into the nucleus so that an E1-deficient adenovirus may also successfully replicate in such a—tumor —cell. In both cases, either by using a YB-1 encoding adenovirus or using an E1-deficient adenovirus in connection with a tumor expressing the YB-1 protein in the nucleus, either due to the particular tumor or by induction of the transfer of YB-1 from the cytoplasma of tumor cells into the nucleus of tumor cells, a selective replication of the adenovirus is possible whereas non-tumor cells and non-tumor tissue are unaffected by this method.

Tumors which express YB-1 in the nucleus are, for example, primary human breast cancer (Bargou, R. C., Jürchott, K., Wagner, C., Bergmann, S., Metzner, S., Bommert, K., Mapara, M. Y., Winzer, K. J., Dietel, M., Dörken, B., and Royer, H. D. Nuclear localization and increased levels of transcription factor YB-1 in primary human breast cancers are associated with intrinsic MDR1 gene expression. Nature Med., 3: 447-450, 1997; Janz, M., Harbeck, N., Dettmar, P., Berger, U., Schmidt, A., Juerchott, K., Schmitt, M., Royer, H-D. Y-box factor YB-1 predicts drug resistance and patient outcome in breast cancer independent of clinically relevant tumor biologic factors HER2, uPA and PAI-1. Int. J. Cancer, 2001 (published online Oct. 10, 2001), human osteosarcoma (Oda, Y., Sakamoto, A., Shinohara, N., Ohga, T., Uchiumi, T., Kohno, K., Tsuneyoshi, M., Kuwano, M., and Iwamoto, Y. Nuclear expression of YB-1 protein correlates with P-glycoprotein expression in human osteosarcoma. Clin. Cancer Res., 4: 3373-3377, 1998), human colorectal carcinomas (Shibao, K. Takano, H., Nakayama, Y., Okazaki, K., Nagata, N., Izumi, H., Uchiumi, T., Kuwano, M., Kohno, K., and Itoh, H. Enhanced coexpression of YB-1 and DNA topoisomerase II alpha genes in human colorectal carcinomas. Int. J. Cancer, 10: 732-737, 1999), ovarian serous adenocarcinoma (Kamura, T. Yahata, H., Amada, S., Ogawa, S., Sonoda, T., Kobayashi, H., Mitsumoto, M., Kohno, K., Kuwano, M., and Nakano, H. Is nuclear expression of y-box binding protein-1 a new prognostic factor in ovarian serous adenocarcinoma. Cancer, 85: 2450-2454, 1999), and non-small cell lung cancer (Nuclear expression of the Y-box binding protein, YB-1, as a novel marker of disease progression in non-small cell lung cancer. Shibahara, K., Sugio, K., Osaki, T., Uchiumi, T., Maehara, Y., Kohno, K., Yasumoto, K., Sugimachi, K., Kuwano, M. Clinical Cancer Res., 7: 3151-3155, 2001).

It is within the skills of the one of the art to administer an adenovirus as described herein to a patient having such tumor and thus in need of such tumor treatment.

A pharmaceutical composition comprising any of the inventive, preferably human, adenoviruses preferably comprises also a pharmaceutically acceptable carrier. Such carrier may be a fluid or a solid. Appropriate fluids are buffers or solutions. Appropriate carriers are known to the one skilled in the art. It is also within the scope of the present invention that the E1-deficient adenoviruses as described herein or the respective pharmaceutical compositions comprising such adenovirus, or the nucleic acid coding for it, or pharmaceutical compositions or medicaments manufactured according to the present invention may comprise further compounds which are known to be also active against tumor cells. Preferably, such compounds are either compounds which mediate the transfer of YB-1 into the nucleus of the cells such as cytostatic agents, or compounds which damage tumor cells such as cytostatic agents or ribozymes.

As used herein, tumors shall be the generic term for tumors, cancers, malignant diseases, cells and tissue(s) exhibiting aberrant growth.

It is also within the scope of the present invention to use the E1-deficient adenovirus comprising a YB-1 encoding DNA sequence or an E1-deficient adenovirus, more particularly in connection with cells exhibiting YB-1 in the nucleus either naturally or by induction, for the selective elimination of such cells for research purposes.

Finally, it is also within the scope of the present invention to use the inventive E1-deficient adenoviruses both encoding or not encoding YB-1, and pharmaceutical composition comprising the same together with substances that damage tumor cells, e.g. cytostatic agents or ribozymes, or with other therapeutic methods such as surgical tumor excision, radiation therapy, chemotherapy, hyperthermia or gene therapy.

This invention claims agents for the E1A-independent replication of recombinant adenoviruses carrying the YB-1 encoding DNA sequence which cause cells to synthesize the recombinant adenoviral YB-1. Hence the invention concerns the use of YB-1 for the E1A-independent replication of adenoviruses. The essence of the invention is among others that a replication-defective E1A adenovirus is able to replicate again in cells as a result of the synthesis of YB-1 which is regulated or controlled by a promotor, such as a tumor-specific promotor, to ensure tumor-specific expression of YB-1 leading to the destruction of the tumor cell.

### BRIEF DESCRIPTION OF THE DRAWING

The invention is now further illustrated by the following figures and examples which are not given to limit the scope of protection but from which further features, embodiments and advantages of the present invention may be taken.

FIG. 1 shows Western Blot analysis of human breast cell line HBL-100 and HBL-100/YB-1;

FIG. 2 shows the effect of E1 deficient Adenovirus on cell lines HBL-100 and HBL-100/YB-1, respectively;

FIG. 3 shows the effect of E1-minus adenovirus (AdlacZ) and YB-1 expressing adenoviruses (AdYB-1) on two different tumor cell lines; and

FIG. 4 shows transmission electron microscopic pictures of microsections of HeLa cells either infected with E1-minus adenovirus (AdlacZ) or YB-1expressing adenovirus (AdYB-1).

### DETAILED DESCRIPTION OF THE INVENTION

### EXAMPLE 1

### Nucleus Located YB-1 allows for E1 Independent Adenoviral Replication

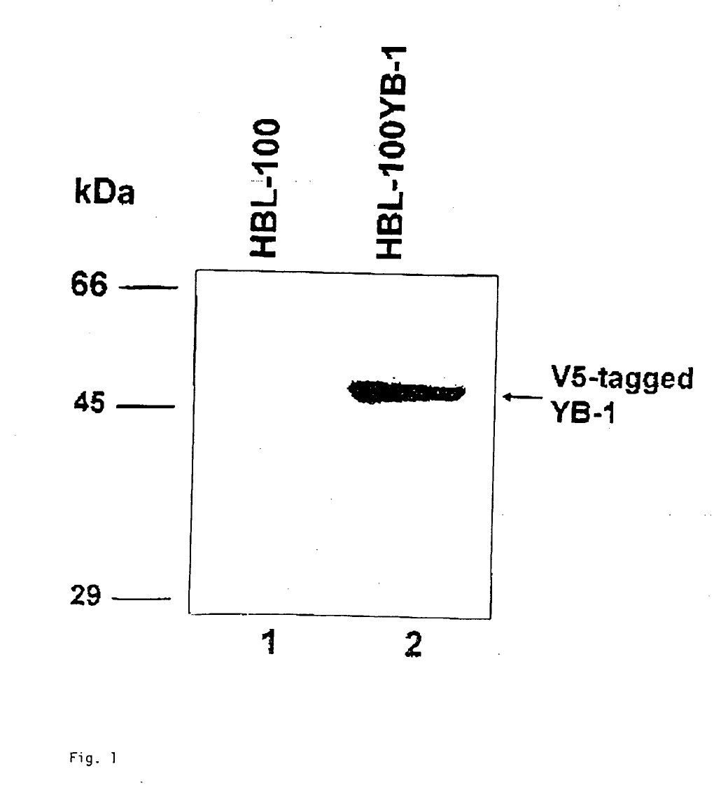

The immortalized epithelial breast cell line HBL-100 was transfected using a vector (pcDNA6/V5-HisB, Invitrogen) into which the cDNA of YB-1 was introduced, by using lipofectamine (Gibco). After selection with blasticidine (5 μl/ml) a stable cell line was isolated and established which over-expressed YB-1 in the nucleus. This newly established cell line was named HBL-100/YB-1. The experimental proof for the successful transfection is shown in FIG. 1. More particularly, FIG. 1 shows a Western blot of a nuclear lysate of both HBL-100 (lane 1) and HBL-100/YB-1 (lane 2). YB-1 was detected by using a V5 antibody (Invitrogen). Only HBL-100/YB-1 over-expressed YB-1 in the nucleus.

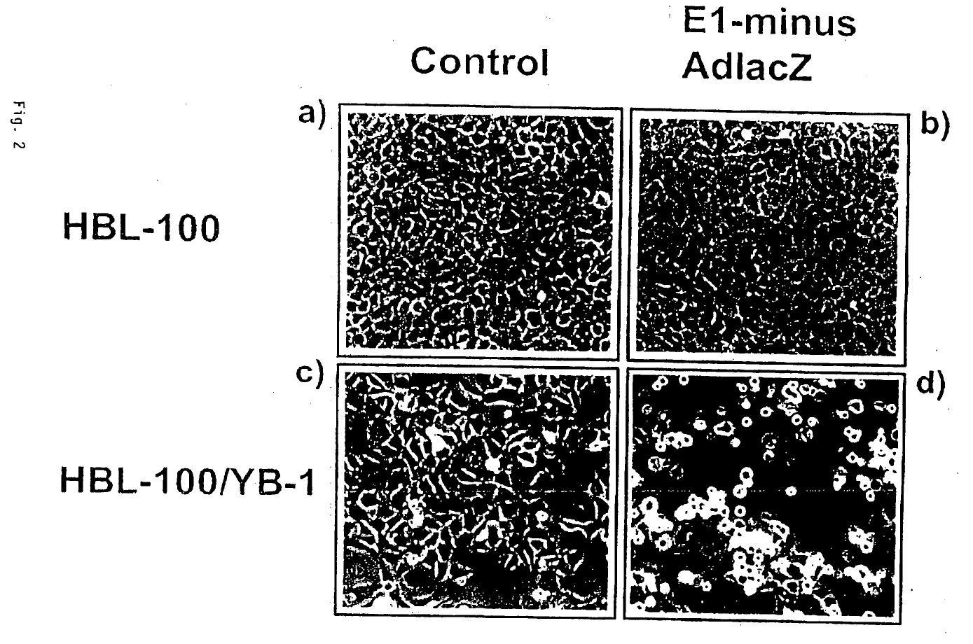

Subsequently, both cell line HBL-100 and HBL-100/YB-1 were transformed with a E1 deleted adenovirus (AdlacZ). This adenovirus is not capable of replicating due to a deletion in the E1 region. The infection was performed by using a multiplicity of infection of 100 for 1 hour at 37° C. After infection the infection medium (Optimen supplemented with 2% FCS) was removed and standardised growth medium added (DMEM with 10% FCS). 3 to 5 days post infection a cytopathic effect (CPE) characterised by a rounding-up of the cells, was observed for the infected HBL-100/YB-1 only as depicted in FIG. 2d). In contrast to this, HBL-100 cells also infected with the E1 deficient adenovirus (E1-minus AdlacZ) did not show such CPE (FIG. 2b)). Uninfected HBL-100 cells and HBL-100/YB-1 cells are depicted in FIG. 2a) and FIG. 2c), respectively.

The CPE observed in connection with cell line HBL-100/YB-1 after infection with an E1-minus recombinant adenovirus proves viral replication within said cell line. This clearly shows that YB-1 allows for an E1-independent viral replication.

### EXAMPLE 2

### Oncolysis of Tumor Cells by an Adenovirus Expressing YB-1

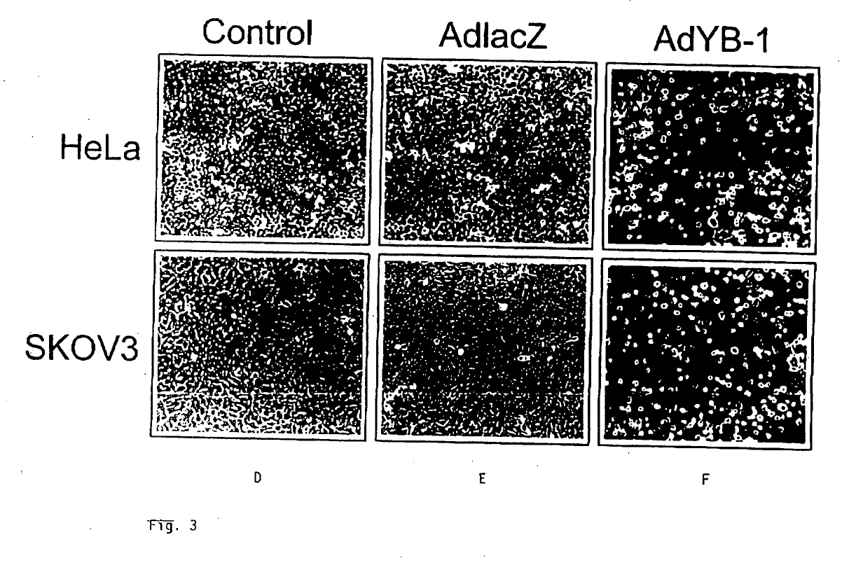

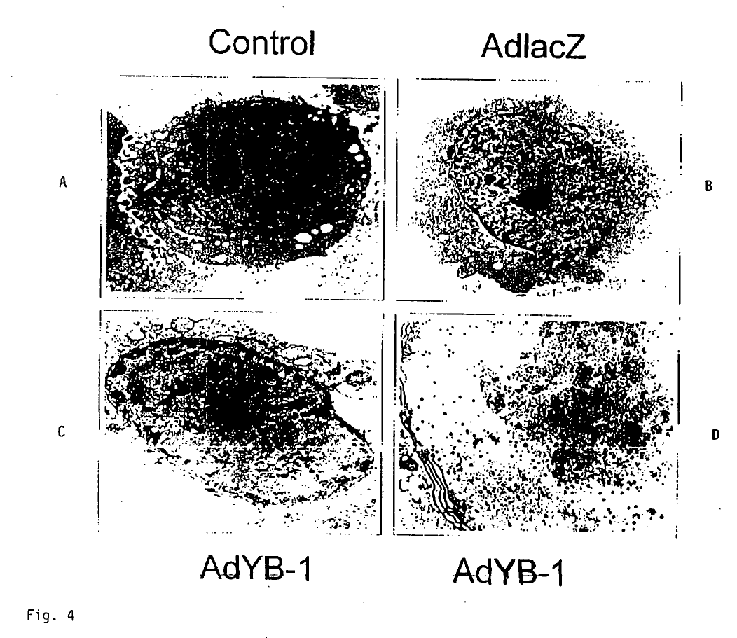

HeLa cells and SkOV3 tumour cells were infected with an E1-minus adenovirus (AdlacZ) and an adenovirus coding for and expressing YB-1 (AdYB-1). The cells were infected with a multiplicity of infection of about 100 for 1 hour at 37° C. After infection the infection medium (Optimen with 2% FKS) was removed and conventional growth medium added (DMEM with 10% FKS). FIGS. 3 and 4 show the result of the experiments.

AdlacZ infected cells did not show any cytopathic effect, i.e. rounding-up of the cells (FIG. 3, B and E), whereas those cells infected with AdYB-1 show a very pronounced cytopathic effect (FIG. 3, C and F). This means that the adenoviral vector AdYB-1 replicates and induces cell lysis.

### EXAMPLE 3

### Formation of Adenoviral Particles in Cell Lines Expressing YB-1 in the Nucleus

This result as shown in example 3 was confirmed in another experiment. HeLa cells were infected with AdYB-1 and AdlacZ (MOI 50). 72 h after infection the cells were prepared for electromicroscopic analysis by standard procedures (2.5% glutaraldehyde embedded in Epon resin and stained with uranyl acetate). Microsections were analysed using a Zeiss EM10CR transmission electron microscope at 60 kV.

Adenoviral particles are only detectable in AdYB-1 infected cells (FIG. 4, C and D at different magnification). HeLa cells not infected with AdlacZ (B) may not be discriminated from control cells which were not infected (A) using morphological criteria. This example as well as example 2 proves that AdYB-1 undergoes a complete viral life cycle once YB-1 is present in the nucleus of a cell which is infected by an E1-minus adenovirus coding for YB-1.

The features of the present invention disclosed in the specification, the claims and/or the drawings may both separately and in any combination thereof be material for realizing the invention in various forms thereof.

What is claimed is:

1. An E1-deficient adenovirus comprising a YB-1 encoding DNA sequence.

2. An E1-deficient adenovirus as in claim 1, wherein said adenovirus is an E1A-deficient adenovirus.

3. A nucleic acid encoding an E1-deficient adenovirus comprising a YB-1 encoding DNA sequence.

4. A nucleic acid as in claim 3, wherein said adenovirus is an E1A-deficient adenovirus.

5. A pharmaceutical composition comprising an E1-deficient adenovirus comprising a YB-1 encoding DNA sequence and at least one pharmaceutically acceptable carrier.

6. A pharmaceutical composition as in claim 5, wherein said adenovirus is an E1A-deficient adenovirus.

7. A method for treatment of tumors, comprising administering to a patient in need thereof an anti-tumor effective amount of a medicament comprising an E1-deficient adenovirus comprising a YB-1 encoding DNA sequence.

8. A method as in claim 7, wherein said adenovirus is an E1A-deficient adenovirus.

9. A method as in claim 7, wherein said medicament is carried on at least one pharmaceutically acceptable carrier.

10. A method as in claim 7, wherein said method includes at least one of:
administration of substances which damage tumor cells;
surgical tumor excision;
radiation therapy;
chemotherapy;
hyperthermia; and
gene therapy.

11. A method as in claim 10, wherein said substances which damage tumor cells are selected from the group consisting of cytostatic agents and ribozymes.

12. A method according to claim 7, wherein said tumor cells are tumor cells which exhibit YB-1 in the nucleus.

13. A method for E1-independent replication of a replication-defective adenovirus comprising
administering to a cell a recombinant adenovirus carrying a YB-1 encoding DNA sequence,
inducing expression of YB-1 in said cell,
causing said adenovirus to replicate in said cell in the presence of YB-1.

14. A method as in claim 13, wherein the adenovirus is a E1-deficient adenovirus.

15. A method as in claim 13, wherein the adenovirus is a E1A-deficient adenovirus.

16. A method as in claim 13, wherein YB-1 is expressed in the nucleus of said cell.

17. A method for E1-independent replication of a replication-defective adenovirus comprising
administering to a cell a replication-defective adenovirus, whereby YB-1 is present in the nucleus of the cell,
causing said adenovirus to replicate in said cell in the presence of YB-1.

18. A method as in claim 17, wherein the presence of YB-1 in the nucleus of the cell is induced preferably by applying heat or by administering cytotoxic substances to the cell.

19. A method as in claim 17, wherein the adenovirus is a E1-deficient adenovirus.

20. A method as in claim 19, wherein the adenovirus is a E1A-deficient adenovirus.
